(19) United States
(12) Patent Application Publication
(10) Pub. No.: US 2025/0261880 A1
(43) Pub. Date: Aug. 21, 2025

(54) IMPLANTABLE OPTICAL SENSOR FOR HEART FAILURE

(71) Applicant: Cardiac Pacemakers, Inc., St Paul, MN (US)

(72) Inventors: Keith R. Maile, New Brighton, MN (US); Michael J. Kane, St Paul, MN (US); Nicholas Anderson, St Paul, MN (US)

(21) Appl. No.: 19/057,391

(22) Filed: Feb. 19, 2025

Related U.S. Application Data
(60) Provisional application No. 63/556,007, filed on Feb. 21, 2024.

(51) Int. Cl. A61B 5/145; A61B 5/00; A61B 5/08; A61B 5/11; A61B 5/1455; A61B 7/02

(52) U.S. Cl. CPC A61B 5/14503; A61B 5/0816; A61B 5/1126; A61B 5/14552; A61B 5/686; A61B 5/7264; A61B 5/7285; A61B 5/746; A61B 7/02; A61B 2560/0443; A61B 2560/0462; A61B 2562/0204; A61B 2562/0219; A61B 2562/16

(57) ABSTRACT

A method includes measuring baseline heart sounds using an ambulatory heart sound monitor, measuring baseline blood oxygen content using an ambulatory optical monitor, measuring new heart sounds, measuring new blood oxygen content, determining a relative increase in the new heart sounds compared to the baseline heart sounds, determining a relative decrease in the new blood oxygen content compared to the baseline blood oxygen content, and generating an alert in response to a combination of both the relative increase and the relative decrease.

### CROSS REFERENCE TO RELATED APPLICATION

This application claims priority to Provisional Application No. 63/556,007, filed Feb. 21, 2024, which is herein incorporated by reference in its entirety.

### TECHNICAL FIELD

Instances of the present disclosure relate to systems, methods, and one or more ambulatory medical devices for sensing various physiological parameters, including blood oxygen content and heart sounds.

### BACKGROUND

Medical devices can be configured to sense physiological parameters and/or provide therapy.

### SUMMARY

In Example 1, a method includes measuring baseline heart sounds using an ambulatory heart sound monitor, measuring baseline blood oxygen content using an ambulatory optical monitor, measuring new heart sounds, measuring new blood oxygen content, determining a relative increase in the new heart sounds compared to the baseline heart sounds, determining a relative decrease in the new blood oxygen content compared to the baseline blood oxygen content, and generating an alert in response to a combination of both the relative increase and the relative decrease.

In Example 2, the method of Example 1, wherein the measuring the new blood oxygen content is performed in response to one or more triggers.

In Example 3, the method of Examples 1 or 2, wherein the measuring the new heart sounds is performed in response to one or more triggers.

In Example 4, the method of Examples 2 or 3, further including detecting activity of a patient, wherein the one or more triggers is the detecting.

In Example 5, the method of Example 4, wherein the detecting is based on an increased respiratory rate.

In Example 6, the method of Example 4, wherein the detecting is based on an output of an acceleration sensor.

In Example 7, the method of any of Examples 1-6, wherein the measuring the baseline heart sounds and the measuring the baseline blood oxygen content is performed while a patient is at rest.

In Example 8, the method of any of Examples 1-7, wherein the relative increase is based on S1 and S3 from the new heart sounds compared to S1 and S3 from the baseline heart sounds.

In Example 9, the method of any of Examples 1-8, further including transmitting the alert to a clinic of a patient.

In Example 10, the method of any of Examples 1-9, wherein the ambulatory heart sound monitor and the ambulatory optical monitor are components of a single implantable medical device.

In Example 11, the method of any of Examples 1-9, wherein the ambulatory heart sound monitor is a component of a first medical device, wherein the ambulatory optical monitor is a component of a second medical device.

In Example 12, the method of Example 11, wherein the first medical device is an external medical device, wherein the second medical device is an implantable medical device.

In Example 13, a computer program product comprising instructions to cause one or more processors to carry out the steps of the method of Examples 1-12.

In Example 14, a computer-readable medium having stored thereon the computer program product of Example 13.

In Example 15, a medical device comprising the computer-readable medium of Example 14.

In Example 16, a system including an ambulatory heart sound monitor configured to measure heart sounds, an ambulatory optical monitor configured to measure blood oxygen content, and circuitry programmed with instructions to cause the circuitry to: determine a relative increase in the heart sounds compared to a heart sounds baseline, determine a relative decrease in the blood oxygen content compared to a blood oxygen content baseline, and generate an alert in response to a combination of both the relative increase and the relative decrease.

In Example 17, the system of Example 16, wherein the circuitry is programmed with the instructions to cause the circuitry to: measure the blood oxygen content in response to one or more triggers.

In Example 18, the system of Example 17, wherein the circuitry is programmed with the instructions to cause the circuitry to: detection of activity of a patient, wherein the one or more triggers is the detection.

In Example 19, the system of Example 18, wherein the detection is based on an increased respiratory rate.

In Example 20, the system of Example 18, wherein the detection is based on an output of an acceleration sensor.

In Example 21, the system of Example 16, wherein the circuitry is programmed with the instructions to cause the circuitry to: measure the heart sounds in response to one or more triggers.

In Example 22, the system of Example 16, wherein the circuitry is programmed with the instructions to cause the circuitry to: measure the heart sounds baseline and measure the blood oxygen content baseline while a patient is at rest.

In Example 23, the system of Example 16, wherein the relative increase is based on S1 and S3 from the heart sounds compared to S1 and S3 from the heart sounds baseline.

In Example 24, the system of Example 16, wherein the circuitry is programmed with the instructions to cause the circuitry to: transmit the alert to a clinic of a patient.

In Example 25, the system of Example 16, wherein the ambulatory heart sound monitor and the ambulatory optical monitor are components of a single implantable medical device.

In Example 26, the system of Example 16, wherein the ambulatory heart sound monitor is a component of a first medical device, wherein the ambulatory optical monitor is a component of a second medical device.

In Example 27, the system of Example 26, wherein the first medical device is an external medical device, wherein the second medical device is an implantable medical device.

In Example 28, the system of Example 16, wherein the ambulatory optical monitor includes a first light emitter arranged to generate a first beam of emitted light, a second light emitter arranged to generate a second beam of emitted light, a light detector arranged to sense backscattered light responsive to the first beam and the second beam, and at least one optical layer comprising a beam steering feature and/or a cross-talk feature.

In Example 29, the system of Example 28, wherein the ambulatory optical monitor further comprises a first wall positioned between the first light emitter and the light detector, wherein the first wall is arranged to at least partially block non-backscattered light.

In Example 30, the system of Example 28, wherein the first light emitter and the second light emitter are part of a single integrated circuit package.

In Example 31, the system of Example 28, wherein the first light emitter and the light detector are part of an optical module positioned within a housing of an implantable medical device.

In Example 32, a method including measuring baseline heart sounds using an ambulatory heart sound monitor, measuring baseline blood oxygen content using an ambulatory optical monitor, measuring new heart sounds, measuring new blood oxygen content, determining a relative increase in the new heart sounds compared to the baseline heart sounds, determining a relative decrease in the new blood oxygen content compared to the baseline blood oxygen content, and generating an alert in response to a combination of both the relative increase and the relative decrease.

In Example 33, the method of Example 32, wherein the measuring the new blood oxygen content is performed in response to detecting activity of a patient.

In Example 34, the method of Example 33, wherein the detecting is based on an output of an acceleration sensor.

In Example 35, the method of Example 32, wherein the measuring the baseline heart sounds and the measuring the baseline blood oxygen content is performed while a patient is at rest.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration depicting an illustrative medical system, in accordance with certain instances of the present disclosure.

FIG. 2 shows a side schematic view of an ambulatory blood oxygen content monitor, in accordance with certain instances of the present disclosure.

FIG. 3 shows a side schematic view of a portion of an optical sensor assembly, in accordance with certain instances of the present disclosure.

FIG. 4 shows a top view of a portion of an optical sensor assembly, in accordance with certain instances of the present disclosure.

FIG. 5A and FIG. 5B show different arrangements of an optical sensor assembly, in accordance with certain instances of the present disclosure.

FIG. 6 shows a schematic of an ambulator heart sounds monitor, in accordance with certain instances of the present disclosure.

FIG. 7 shows an implantable medical device, in accordance with certain instances of the present disclosure.

FIG. 8 shows a block diagram of a method, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Instances of the present disclosure utilize ambulatory devices for monitoring blood oxygen content and heart sounds. Blood oxygen content and heart sounds can be monitored and used to detect heart failure.

The description below first outlines a system in which one or more ambulatory medical devices are used to monitor blood oxygen content and heart sounds. The description then describes an example of an ambulatory blood oxygen content monitor followed by a description of an ambulatory heart sound monitor. In certain instances, the ambulatory blood oxygen content monitor and the ambulatory heart sound monitor are parts of the same ambulatory medical device (e.g., the same implantable medical device). In other instances, the ambulatory blood oxygen content monitor and the ambulatory heart sound monitor are separate ambulatory medical devices (e.g., an implantable medical device and a separate external medical device).

The blood oxygen content and heart sounds measured by the one or more the ambulatory medical devices can be compared to respective baseline blood oxygen content and baseline heart sounds to determine or predict aspects of heart failure of a patient. Under certain circumstances, when the patient's blood oxygen content has decreased and the patient's heart sounds have increased, an alert can be generated to provide notice of heart failure, and the alert can be transmitted to a clinic.

In certain instances, to save power, measuring the blood oxygen content and/or the heart sounds is performed in response to one or more triggers (e.g., triggers indicating patient activity such as respiratory rate and/or an output of an activity sensor like an acceleration sensor).

In certain instances, obtaining baseline blood oxygen content and baseline heart sounds is performed by the one or more ambulatory medical devices while a patient is at rest. In certain instances, the hearts sounds used to determine heart failure are S1 heart sounds and S3 heart sounds.

These and other aspects of measuring and using blood oxygen content and heart sounds to detect heart failure are described below.

### Medical Device System

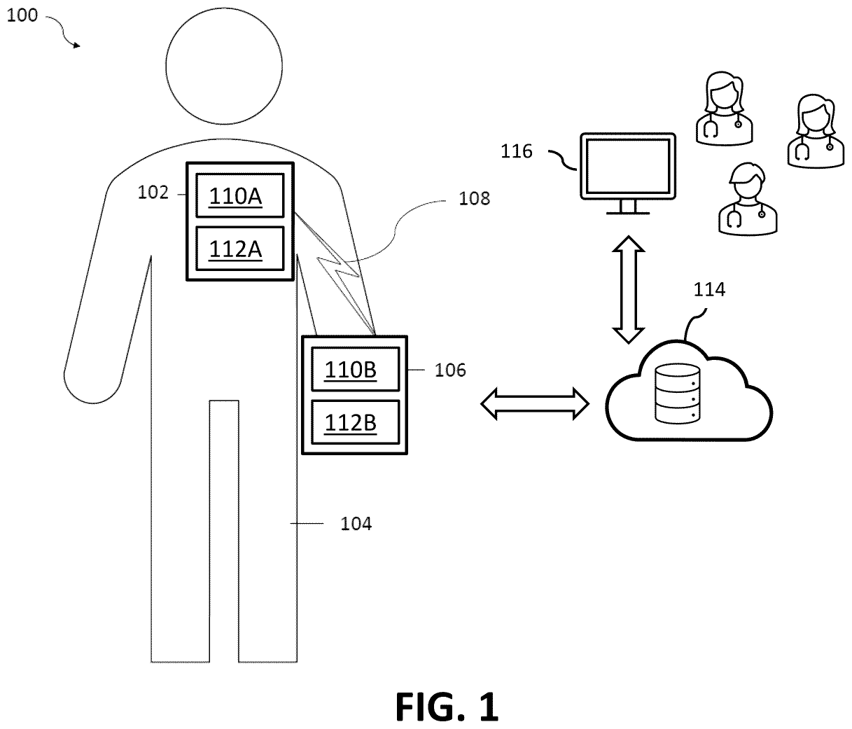

FIG. 1 shows an illustrative medical device system 100, which includes an implantable medical device 102 (hereinafter an “IMD” for brevity) configured to be implanted within the body of a subject 104 and an external medical device 106 (EMD). In the example of FIG. 1, the IMD 102 and the EMD 106 are configured to communicate with one another over a communication link 108.

The IMD 102 may be implanted subcutaneously within an implantation location or pocket in the patient's chest or abdomen and may be configured to monitor (e.g., sense and/or record) one or more physiological parameters. The IMD 102 may be an implantable cardiac monitor (ICM) (e.g., an implantable diagnostic monitor (IDM), an implantable loop recorder (ILR)) configured to record physiological parameters such as, for example, one or more cardiac electrical signals, blood oxygen content, heart sounds, heart rate, blood pressure measurements, and/or the like.

The EMD 106 may be a device that is configured to be portable with the subject 104, e.g., by being integrated into a vest, belt, harness, sticker; placed into a pocket, a purse, or a backpack; carried in the subject's hand; and/or the like, or otherwise operatively (and/or physically) coupled to the subject 104. The EMD 106 may be configured to receive data from the IMD 102 and/or monitor physiological parameters associated with the subject 104 and/or provide therapy to the subject 104. In certain instances, the EMD 106 is a cell phone or other device with a user interface that can be used to view aspects of the physiological parameters recorded by the IMD 102.

The IMD 102 can include an ambulatory blood oxygen content monitor 110A and an ambulatory heart sound monitor 112A. The EMD 106 can have its own ambulatory blood oxygen content monitor 110B and an ambulatory heart sound monitor 112B.

Certain physiological data can be communicated (e.g., communicated wirelessly using an antenna) to one or more other components within the system 100. For example, physiological data can be communicated from the IMD 102 to the EMD 106, a computing system 108 (e.g., a server), and/or a remote computing device 110 (e.g., a computing device at a clinic).

### Ambulatory Blood Oxygen Content Monitor

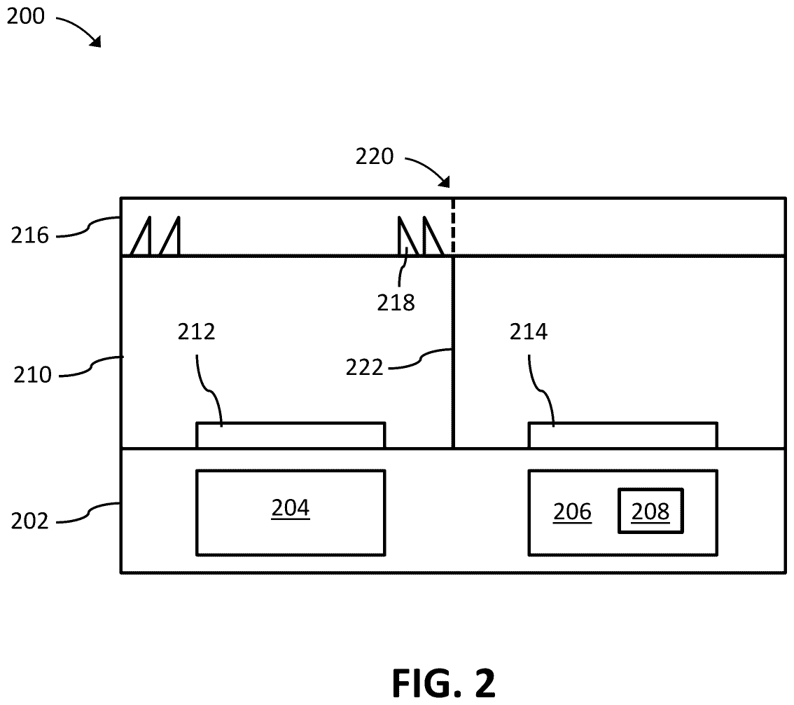

FIG. 2 shows a schematic of an ambulatory blood oxygen content monitor 200 that can be used in an implantable medical device (such as the IMD 102 of FIG. 1) and an external medical device (such as the EMD 106 of FIG. 1). The ambulatory blood oxygen content monitor 200 can utilize one or more optical sensor devices to measure physiological parameters such as oxygen saturation in blood. In certain instances, the output of the ambulatory blood oxygen content monitor 200 is a photoplethysmography (PPG) signal that indicates, over time, a person's blood oxygen content.

In instances where the ambulatory blood oxygen content monitor 200 is part of an external device, the ambulatory blood oxygen content monitor 200 can be placed on a person's body part (e.g., finger, ear lobe) so that light is passed through the body part to an optical sensor. The person's blood oxygen content (or oxygen saturation) can be estimated based on how the light is absorbed as the light passes through blood flowing in the body part.

In instances where the ambulatory blood oxygen content monitor 200 is part of an implantable medical device, the ambulatory blood oxygen content monitor 200 can be incorporated with other types of monitors such as monitors that measure a person's cardiac electrical signals, heart sounds, etc.

The features shown in FIG. 2 are arranged for use in an implantable medical device. However, features such as light emitters and light detectors could be rearranged with respect to each other for use with an external medical device.

The ambulatory blood oxygen content monitor 200 includes circuitry 202, which can include one or more processors 204, memory 206, and executable instructions 208. The memory 206 stores the executable instructions 208 for causing the one or more processors 204 to perform functions such as controlling components of the ambulatory blood oxygen content monitor 200. The executable instructions 208 may include, for example, computer code, machine-useable instructions, and the like such as, for example, program components capable of being executed by the one or more processors 204 (e.g., microprocessors). In certain instances, the one or more processors 204, memory 206, and instructions 208 are part of a controller such as an application specific integrated circuit (ASIC), field-programmable gate array (FPGA), and/or the like to carry out the functions of the ambulatory blood oxygen content monitor 200.

The ambulatory blood oxygen content monitor 200 can include an optical module 210. The optical module 210 includes one or more emitters 212 (hereinafter the “emitter 212”) and one or more detectors 214 (hereinafter the “detector 214”). The optical module 210 includes one or more optical layers 216 (hereinafter the “optical layer 216”) that allow light to pass through (e.g., like a window) but that protect the emitter 212 and the detector 214 from the environment external to the optical module 210. As shown in FIG. 2, both the emitter 212 and the detector 214 have a top surface (e.g., an emitting surface for the emitter 212 and a detecting surface for the detector 214) that faces a bottom surface of the optical layer 216. In certain instances, the optical layer 216 (with its one or more layers) creates a common window for light to both exit and enter the optical module 210—whereas in other instances, the optical layer 216 comprises separate windows (e.g., one or more layers for light exiting and one or more layers for light entering the optical module 210). In either instance, as discussed below, one or more portions of the optical layer 216 can have separate properties or features (e.g., beam steering/filtering/blocking properties or features) than the other portions of the optical layer 216. Regardless of whether one window or multiple windows are used, the window(s) can comprise glass, sapphire, and the like.

As described in more detail below, the emitter 212 (e.g., one or more light sources such as light-emitting diodes) can be selectively powered such that the emitter 212 emits light out of the optical module 210 and towards a patient's tissue. At least some light will be reflected back (e.g., backscattered light) to the optical module 210 and sensed by the detector 214 (e.g., one or more light sensors such as photodetectors or another type of light sensor). The sensed backscattered light can be used by the ambulatory heart sound monitor 200 to measure physiological parameters such as the patient's blood oxygen content (e.g., SpO2).

There are multiple challenges with emitting and sensing backscattered light with optical sensing devices. One challenge is directing enough backscattered light to the detector 214 due to light being reflected back in directions that are misaligned with the detector 214 such that the reflected light cannot be sensed by the detector 214. To help address this challenge, the optical module 210 can incorporate one or more beam steering features 218.

One example beam steering feature 218 is a lens (e.g., Fresnel lens, contact lens). A lens can be incorporated into an optical layer adjacent to the emitter 212. The lens can direct or focus light in a direction that increases the amount of backscattered light that will ultimately be reflected back to and sensed by the detector 214. Because lenses such as a Fresnel lens can have a rough surface, the lens can be covered by a layer of another optical material to create a smooth outer surface for the optical module 210. Another example beam steering feature 218 is an optical grating designed to direct light in a direction that increases the amount of backscattered light that will be reflected back to and sensed by the detector 214.

Another challenge with optical sensing devices is that non-backscattered light can interfere with the backscattered light. For example, not all light from the emitter 212 may exit the optical module 210 which can cause interference with the backscattered light. To help address this challenge, the optical module 210 can incorporate one or more cross-talk features into the optical layer 216. The example cross-talk features described below are not mutually exclusive and can be used with each other. Similarly, the optical module 210 can use both one or more cross-talk features and one or more beam steering features 218.

As one example of a cross-talk feature, the material and/or dimensions (e.g., thickness) of the optical layer 216 and/or the relative position of the optical layer 216 to the emitter 212 and the detector 214 can be selected to reduce the risk of cross-talk. The relative position of the optical layer 216, the emitter 212, and the detector 214 can be selected such that light is less likely to reflect back towards the emitter 212. Put another way, selection of the relative position of the optical layer 216, the emitter 212, and the detector 214 can help control the amount of light that reflects internally due to its angular relationship with the critical angle of the optical layer 216.

As another example of a cross-talk feature, the optical layer 216 can include a section 220 (represented by a dashed line in FIG. 2) that wholly or partially blocks light that is internally reflected in the optical layer 216. Some emitted light may get “trapped” in the optical layer 216 and get directed towards the portion of the optical layer 216 adjacent to (e.g., directly above) the detector 214. The section 220 can act as a wall or block within the optical layer 216 to help prevent light that is internally reflected within the optical layer 216 from interfering with backscattered light reflected towards the detector 214. For example, the section 220 can include a dark region that is diffused into the optical layer 216. This section 220 of dark material can act like a wall in which the internally reflected light cannot pass through. Additionally or alternatively, the optical module 210 can include one or more walls 222 positioned between the emitter 212 and the detector 214 such that emitted light does not travel within the optical module 210 from the emitter's housing portion to the detector's housing portion.

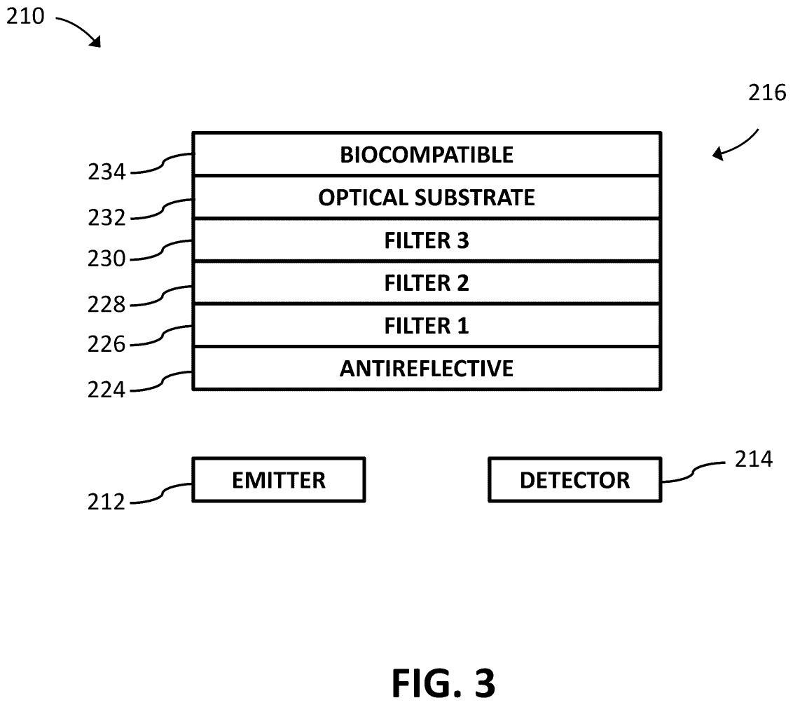

FIG. 3 shows an example layer structure for helping to prevent light from interfering with the backscattered light. As shown in the side view of FIG. 3, the optical layer 216 comprises multiple layers. The number and arrangement of the layers can vary from what is shown in FIG. 3. The layers can include a layer of antireflective material 224 closest to the emitter 212 and the detector 214. One or more optical filter layers 226, 228, and 230 can be positioned between the antireflective layer 224 and an optical substrate 232. The filter layers 226-230 can be designed to help prevent light generated from sources external to the optical module 210 from reaching the detector 214 and/or tune the light generated by the emitter 212. If the optical module 210 is oriented such that it is close to and facing a patient's skin, light such as sunlight can pass through the patient and reach the optical module 210. As such, the filter layers 226-230 can each be designed/selected to filter out certain undesired wavelengths of light (e.g., sunlight, fluorescent light) that are most likely to reach the optical module 210. Additionally or alternatively, one or more optical filters can be coupled directly to the emitter 212 and/or the detector 214. As previously noted, one portion of the optical layer 216 can have different features (e.g., filter(s), doping) than other portions of the optical layer 216. As such, the portion of the optical layer 216 directly above the emitter 212 can use one or more types of filters that are different than the portion of the optical layer 216 directly above the detector 214.

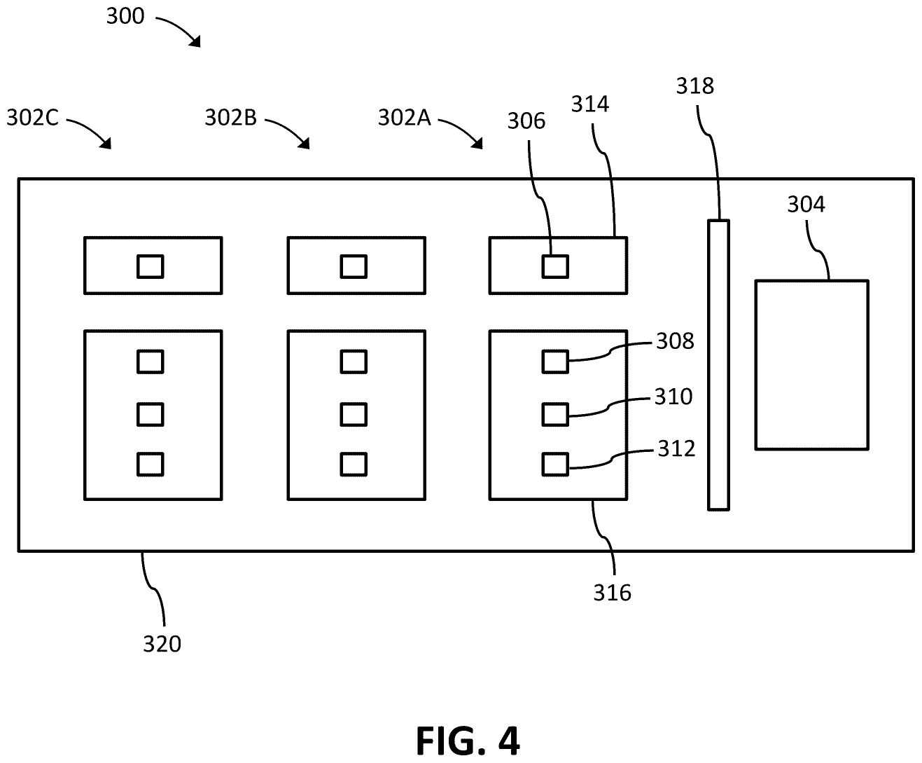

FIG. 4 shows a top view of part of an optical sensor assembly or optical module 300 (hereinafter the “optical module 300” for brevity), which can be incorporated into an ambulatory blood oxygen content monitor and ultimately into a medical device. As such, the features shown in FIGS. 2 and 3 can be used with the features shown in FIG. 4.

The optical module 300 of FIG. 4 includes multiple sets of emitters. For example, the optical module 300 includes a first set 302A of emitters, a second set 302B of emitters, and a third set 302C of emitters. The optical module 300 also includes one or more detectors 304 (e.g., a photodetector or other type of light sensor).

Each set of emitters can include an infrared emitter 306 and one or more visible light emitters such as a blue light emitter 308, a green light emitter 310, and/or a red light emitter 312. Although FIG. 4 shows the infrared emitter 306 as part of one integrated circuit package 314 and the visible light emitters as part of another integrated circuit package 316, the emitters can all be integrated into a single package or separate packages. The optical module 300 also includes a wall 318, which helps prevent light from the emitters from interfering with backscattered light directed towards the detector 304. Each of the components mentioned above can be attached to a substrate 320 such as a printed circuit board or flexible circuit board. When completed, the substrate 320 can be positioned within an IMD or within a separate module that is then incorporated into an IMD. In some instances, the substrate 320 is a single substrate to which all of the above-mentioned components are attached, however, in other instances the components can be attached to separate substrates.

During use, the emitters can be selectively powered to emit light. In some instances, the emitters are selectively powered to determine which emitter or set of emitters provides the best amount (or highest quality) of backscattered light to the detector 304. Because each emitter has a different position relative to the detector 304, each emitter will create a different light path from the respective emitter to the detector 304. The emitters can be cycled through on a 1-by-1 basis or a set-by-set basis or some combination thereof to determine which emitter(s) should be used for measuring and recording physiological parameters. For example, one or more emitters in the first set 302A of emitters can be powered first, followed by the second set 302B, followed by the third set 302C. Circuitry or logic in an IMD, EMD, or another device can determine which set or emitters provides the best amount (or highest quality) of backscattered light to the detector 304 compared to the other sets. For example, the emitter that results in the highest measured amplitude can be ultimately selected for measuring and recording physiological parameters. In instances with multiple detectors 304, different emitter-detector combinations can be tested to see which results in the best amount (or highest quality) of sensed backscattered light.

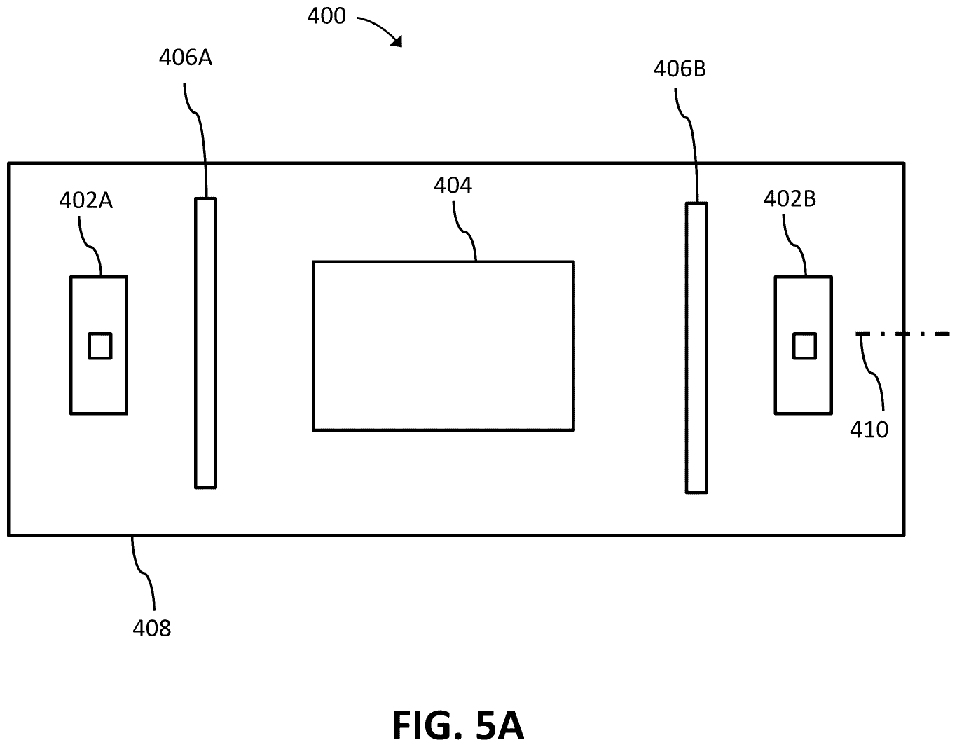

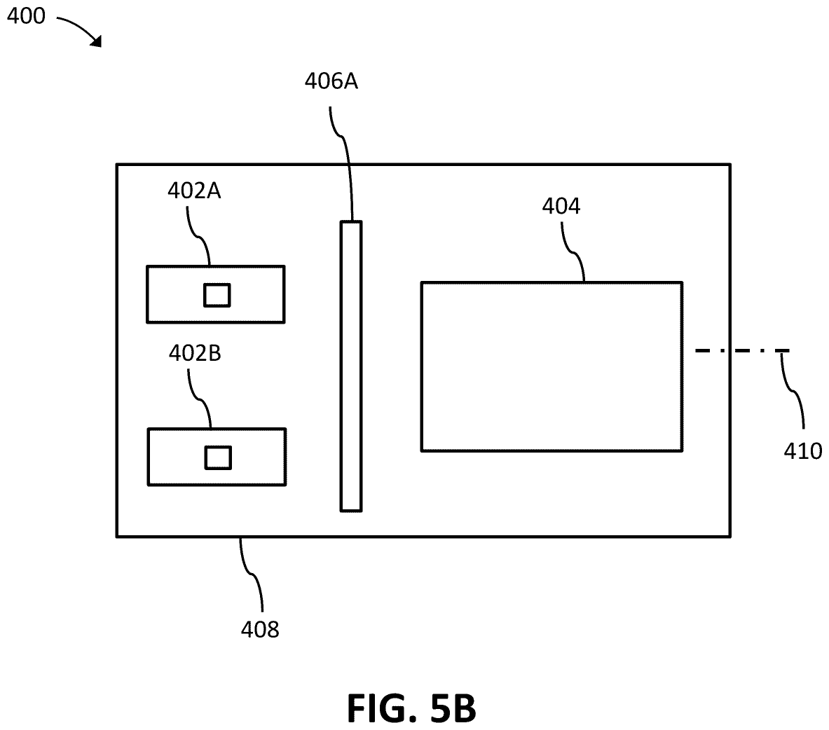

FIGS. 5A and 5B show other example optical modules, which can be incorporated into ambulatory blood oxygen content monitor and ultimately into a medical device. As such, the features shown in FIGS. 2-4 can be used with the features shown in FIGS. 5A and 5B.

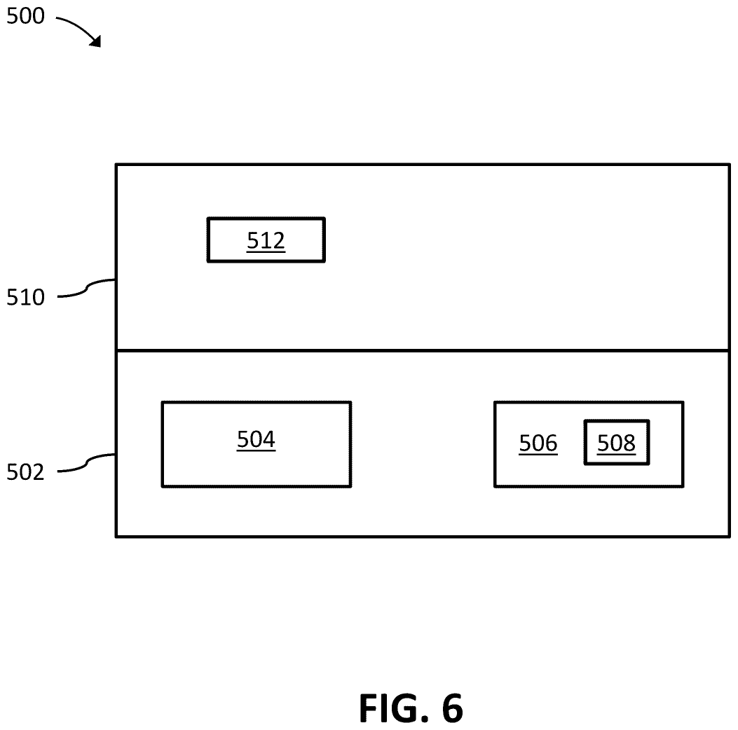

Starting with FIG. 5A, an optical module 400 includes a first emitter 402A, a second emitter 402B, one or more detectors 404, a first wall 406A, and a second wall 406B-all of which can be coupled to one or more substrates 408. Although only two emitters and one detector are shown in FIG. 6A, additional emitters and detectors can be used.

In certain embodiments, the first emitter 402A is a light emitting diode (LED) that is designed to emit red light (e.g., wavelengths around 620-750 nm) while the second emitter 402B is an LED that is designed to emit infrared light (e.g., wavelengths around 800 nm to 1 mm). The first emitter 402A and the second emitter 402B can be spaced from the detector 404 along a longitudinal axis 410 of the optical module 400 or an IMD. In the example of FIG. 5A, the detector 404 is positioned between the first emitter 402A and the second emitter 402B, and the respective walls 406A and 406B are each positioned between the detector 404 and one of the emitters 402A and 402B. In some instances, an edge of the first emitter 402A and an edge of the second emitter 402B are positioned 3-6 mm (e.g., 4 mm, 5 mm) from an edge of the detector 404 (e.g., along the longitudinal axis 410 such as a central longitudinal axis of the optical module 400). This range of distances has been found to be sufficient for the detector 404 to receive enough backscattered light from tissue. Although each of the components mentioned above are shown as being coupled to a single substrate 408 (e.g., a single printed circuit board or a single flexible circuit board), the components can be attached to different substrates. Because medical devices are designed to be compact and because the emitters and detector(s) should be spaced from each other, the space between the emitters and detector(s) can be used for other components for the optical module 400. As such, respective emitters and detector(s) can be coupled to separate substrates so that other components can be more easily positioned between the emitters and detector(s).

FIG. 5B shows the optical module 400 with most of the same components shown in FIG. 5A but rearranged for a more compact design. Because the first emitter 402A and the second emitter 402B are arranged on the same side of the detector 404, the overall length of the optical module 400 can be reduced compared to the design of FIG. 5A. In some instances, an edge of the first emitter 402A and an edge of the second emitter 402B are positioned 3-6 mm (e.g., 4 mm, 5 mm) from an edge of the detector 404 (e.g., along the longitudinal axis 410 such as a central longitudinal axis of the optical module 400).

### Ambulatory Heart Sounds Monitor

FIG. 6 shows a schematic of an ambulatory heart sounds monitor 500 that can be used in an implantable medical device (such as the IMD 102 of FIG. 1) and an external medical device (such as the EMD 106 of FIG. 1).

Heart sounds are recurring mechanical signals associated with cardiac vibrations from and blood flow through the heart with each cardiac cycle. Different heart sounds can be separated and classified according to activity associated with the vibrations and blood flow. The first heart sound (commonly referred to as “S1”) is the vibrational sound made by the heart during closure of the atrioventricular valves. The second heart sound (S2) is the beginning of diastole and is made by the aortic and pulmonary valves. The third and fourth heart sounds (S3 and S4) are related to filling pressures of the left ventricle during diastole.

The ambulatory heart sounds monitor 500 can be designed to measure heart sounds. In instances where the ambulatory heart sounds monitor 500 is part of an external device, the ambulatory heart sounds monitor 500 can be part of a device such as a smart phone or another type of device that can be placed in a vest, belt, harness, sticker, pocket, purse, backpack, or otherwise coupled to a person. In instances where the ambulatory heart sounds monitor 500 is part of an implantable medical device, the ambulatory heart sounds monitor 500 can be incorporated with other types of monitors such as monitors that measure a person's cardiac electrical signals, blood oxygen content, etc.

The ambulatory heart sounds monitor 500 includes circuitry 502, which can include one or more processors 504, memory 506, and executable instructions 508. The memory 506 stores the executable instructions 508 for causing the one or more processors 504 to perform functions such as controlling components of the ambulatory heart sounds monitor 500. The executable instructions 508 may include, for example, computer code, machine-useable instructions, and the like such as, for example, program components capable of being executed by the one or more processors 504 (e.g., microprocessors). In certain instances, the one or more processors 504, memory 506, and instructions 508 are part of a controller such as an application specific integrated circuit (ASIC), field-programmable gate array (FPGA), and/or the like to carry out the functions of the ambulatory heart sounds monitor 500.

The ambulatory heart sounds monitor 500 can include a heart sound module 510, which includes a heart sounds sensor 512. The heart sounds sensor 512 can be a sensor configured to detect heart sounds and to produce one or more signals as a function of the detected heart sounds. In certain instances, the heart sounds sensor 512 is an accelerometer, a microphone, or other type of transducer configured to produce an electronic signal based on a detected force, acceleration, and/or pressure.

### Implantable Medical Device

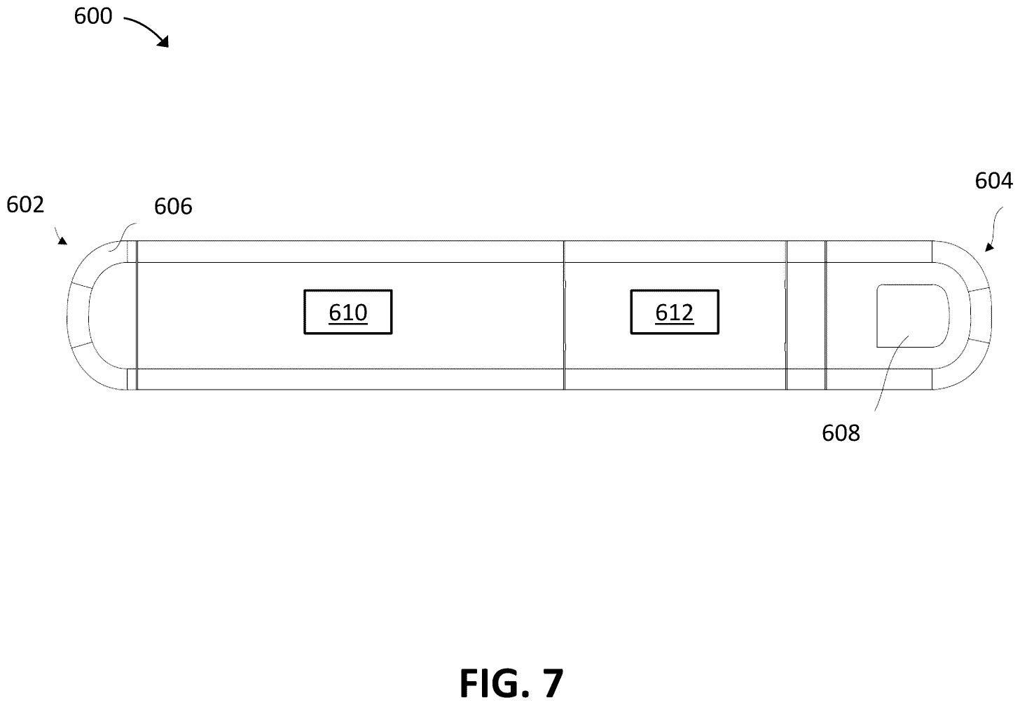

FIG. 7 is a side view of an implantable medical device 600 (IMD). The IMD 600 may be, or may be similar to, the IMD 102 depicted in FIG. 1 and may be used in the system 100 of FIG. 1. The IMD 600 can include features described in U.S. patent application Ser. No. 15/242,470 (which is hereby incorporated by reference in its entirety) with the addition of an ambulatory blood oxygen content monitor and/or an ambulatory heart sound monitor described herein.

The IMD 600 includes an external housing that extends between a first end 602 and a second end 604. When assembled, the external housing can create a hermetically sealed enclosure. The IMD 600 can include one or more electrodes 606, 608 for sensing electrical activity of a patient. The IMD 600 can also include features such as an antenna for communicating with external devices, a battery for powering electrical components of the IMD 600, and circuitry (e.g., one or more computing devices as described further herein), among other features.

FIG. 7 shows the IMD 600 having both an ambulatory blood oxygen content monitor 610 and an ambulatory heart sound monitor 612 such as the monitors described herein. In instances where a device such as the IMD 600 includes both an ambulatory blood oxygen content monitor and an ambulatory heart sound monitor, the outputs (e.g., sensor signals) of the monitors may be communicated to a shared set of circuitry programmed to process and analyze outputs from both monitors and make determinations (e.g., whether to generate an alert, when to transmit an alert).

### Methods

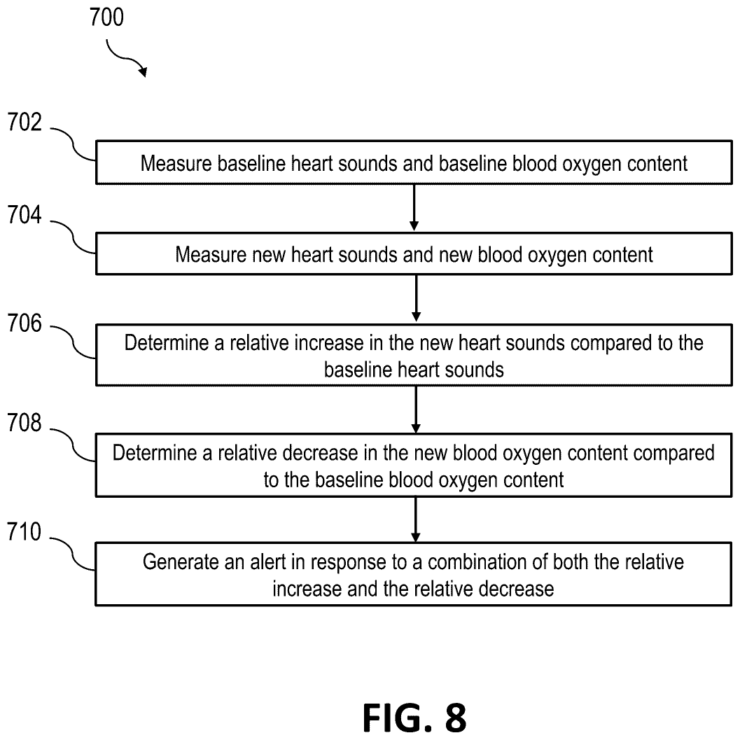

FIG. 8 is a block diagram of a method 700 for use with an ambulatory blood oxygen content monitor and an ambulatory heart sound monitor. The monitors may be part of the same device or may be parts of separate devices. As noted above, blood oxygen content and heart sounds can be monitored and used to detect heart failure. The method 700 can be used to assist with determining the existence of indicators that predict heart failure.

The method 700 includes measuring baseline heart sounds using an ambulatory heart sound monitor and measuring baseline blood oxygen content using an ambulatory blood oxygen content monitor (block 702 in FIG. 8). The baseline heart sounds and the baseline blood oxygen content can be measured to determine a patient's baseline health for later comparisons.

In certain instances, the baseline heart sounds include a strip of time-series data comprising one or more cardiac cycles and the responsive heart sound amplitudes during the cardiac cycle(s). The baseline heart sounds can include values indicating amplitude peaks or valleys for certain heart sounds (e.g., S1 heart sounds, S2 heart sounds, S3 heart sounds, S4 heart sounds). Additionally or alternatively, the baseline heart sounds can include values such as an average value, a median value, or other values for certain heart sounds such as S1, S2, S3, and S4 heart sounds.

In certain instances, the baseline blood oxygen content includes a strip of time-series PPG data. The baseline blood oxygen content can include percentage values (e.g., 93%, 94%, 95%, 96%, 97%, and so on) indicating an oxygen saturation percentage at a given point of time. Additionally or alternatively, the baseline blood oxygen content can include values such as an average value, a median value, or other values indicating blood oxygen content.

In certain instances, measuring the baseline blood oxygen content and measuring the baseline heart sounds is performed while a patient is at rest. Determining whether the patient is at rest can be based on a respiratory rate and/or an output of an activity sensor like an acceleration sensor. In some instances, the measuring the baseline blood oxygen content and measuring the baseline heart sounds is performed while the patient is in a clinic.

Once a baseline is established, the ambulatory heart sound monitor and the ambulatory oxygen content monitor can continue to monitor the patient's heart sounds and blood oxygen content. As such, the method 700 further includes measuring new heart sounds and measuring new blood oxygen content (block 704 in FIG. 8). In certain instances, the baselines are measured using a different monitor than that used to measure the new heart sounds and new blood oxygen content. For example, the baselines could be measured using a clinic device, and the new heart sounds and new blood oxygen content could be measure by the ambulatory heart sound monitor and the ambulatory oxygen content monitor. Regardless of which device measure the baselines, the baselines can be considered control data that will be unique for each different patient.

Next, the new heart sounds and the new blood oxygen content are compared to the respective baseline values. More particularly, the method 700 includes determining a relative increase in the new heart sounds compared to the baseline heart sounds (block 706 in FIG. 8) and determining a relative decrease in the new blood oxygen content compared to the baseline blood oxygen content (block 708 in FIG. 8).

Heart failure can make it difficult for a person to exercise (including walking) due to increased venous blood flow return into a relaxing and stiff left ventricle. This results in a large and rapid increase in pulmonary capillary wedge pressure (PCWP), which makes it difficult for a patient to breath when exercising. It has been found that an increase in PCWP can be determined by analyzing heart sounds—in particular, S1 heart sounds and S3 heart sounds. An increase in PCWP results in an increase in S1 heart sounds because of right atrial ejection and an increase in S3 heart sounds because of left ventricle filling. As such, S1 heart sounds and S3 heart sounds can be compared to baseline S1 and S3 heart sounds to determine a potential indicator of heart failure.

To further predict the likelihood of heart failure, the new blood oxygen content can be compared to the baseline blood oxygen content. If the new blood oxygen content has decreased, this can indicate that the patient is experiencing shortness in breath due to the lower blood oxygen content. As noted above, shortness in breath can be a result of an increase in PCWP.

In certain instances, to save power, the new blood oxygen content and/or the new heart sounds are not measured continuously. For example, the new blood oxygen content and/or the new heart sounds may be measured in response to one or more triggers (e.g., trigger events). As such, the ambulatory blood oxygen monitor and/or the ambulatory heart sounds monitor may be activated (e.g., powered on) in response to one or more triggers. One example trigger is when patient activity is detected. Patient activity could be detected based on output of an activity sensor such as an acceleration sensor that is part of an implantable medical device, external medical device, or another device. In another approach, patient activity could be detected by a patient's respiratory rate.

The method 700 further includes generating an alert in response to a combination of both the relative increase and the relative decrease (block 710 in FIG. 8). In certain instances, the relative increase and the relative decrease must be a minimum threshold (e.g., a minimum difference between the new heart sounds and the baseline and/or the new blood oxygen content and the baseline), and the threshold could be customized by patient. Once an alert is generated, the alert can be transmitted to the patient's clinic.

In certain instances, the comparison between new heart sounds and the baseline and/or the new blood oxygen content and the baseline is performed by a medical device such as those described herein. For example, if both the ambulatory heart sounds monitor and the ambulatory blood oxygen content monitor are part of the same medical device, that device could be programmed (e.g., via its circuitry) to perform the comparison and generate the alert, along with other functions. In other examples, the underlying heart sounds and blood oxygen content are transmitted to a different device (e.g., a person's smart phone), and the smart phone is programmed (e.g., via a phone application) to perform the comparison, generate the alert, etc. In instances where the ambulatory heart sounds monitor and the ambulatory blood oxygen content monitor are separate devices, the respective devices could perform their respective comparisons and one or a different device can be programmed to perform the comparison, generate the alert, etc.

Regardless of which device performs which functions, the ambulatory heart sounds monitor and the ambulatory blood oxygen content monitor allow for a patient's heart health to be monitored without the patient needing to periodically go to a clinic for testing. Instead, the ambulatory heart sounds monitor and the ambulatory blood oxygen content monitor can track relevant data and alert a clinic (and/or the patient) in the event the heart sounds and blood oxygen content indicate potential heart failure.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
an ambulatory heart sound monitor configured to measure heart sounds;
an ambulatory optical monitor configured to measure blood oxygen content;
circuitry programmed with instructions to cause the circuitry to:
determine a relative increase in the heart sounds compared to a heart sounds baseline,
determine a relative decrease in the blood oxygen content compared to a blood oxygen content baseline, and
generate an alert in response to a combination of both the relative increase and the relative decrease.

2. The system of claim 1, wherein the circuitry is programmed with the instructions to cause the circuitry to: measure the blood oxygen content in response to one or more triggers.

3. The system of claim 2, wherein the circuitry is programmed with the instructions to cause the circuitry to: detection of activity of a patient, wherein the one or more triggers is the detection.

4. The system of claim 3, wherein the detection is based on an increased respiratory rate.

5. The system of claim 3, wherein the detection is based on an output of an acceleration sensor.

6. The system of claim 1, wherein the circuitry is programmed with the instructions to cause the circuitry to: measure the heart sounds in response to one or more triggers.

7. The system of claim 1, wherein the circuitry is programmed with the instructions to cause the circuitry to: measure the heart sounds baseline and measure the blood oxygen content baseline while a patient is at rest.

8. The system of claim 1, wherein the relative increase is based on S1 and S3 from the heart sounds compared to S1 and S3 from the heart sounds baseline.

9. The system of claim 1, wherein the circuitry is programmed with the instructions to cause the circuitry to: transmit the alert to a clinic of a patient.

10. The system of claim 1, wherein the ambulatory heart sound monitor and the ambulatory optical monitor are components of a single implantable medical device.

11. The system of claim 1, wherein the ambulatory heart sound monitor is a component of a first medical device, wherein the ambulatory optical monitor is a component of a second medical device.

12. The system of claim 11, wherein the first medical device is an external medical device, wherein the second medical device is an implantable medical device.

13. The system of claim 1, wherein the ambulatory optical monitor comprises:
a first light emitter arranged to generate a first beam of emitted light;
a second light emitter arranged to generate a second beam of emitted light;
a light detector arranged to sense backscattered light responsive to the first beam and the second beam; and
at least one optical layer comprising a beam steering feature and/or a cross-talk feature.

14. The system of claim 13, wherein the ambulatory optical monitor further comprises a first wall positioned between the first light emitter and the light detector, wherein the first wall is arranged to at least partially block non-backscattered light.

15. The system of claim 13, wherein the first light emitter and the second light emitter are part of a single integrated circuit package.

16. The system of claim 13, wherein the first light emitter and the light detector are part of an optical module positioned within a housing of an implantable medical device.

17. A method comprising:
measuring baseline heart sounds using an ambulatory heart sound monitor;
measuring baseline blood oxygen content using an ambulatory optical monitor;
measuring new heart sounds;
measuring new blood oxygen content;
determining a relative increase in the new heart sounds compared to the baseline heart sounds;
determining a relative decrease in the new blood oxygen content compared to the baseline blood oxygen content; and
generating an alert in response to a combination of both the relative increase and the relative decrease.

18. The method of claim 17, wherein the measuring the new blood oxygen content is performed in response to detecting activity of a patient.

19. The method of claim 18, wherein the detecting is based on an output of an acceleration sensor.

20. The method of claim 17, wherein the measuring the baseline heart sounds and the measuring the baseline blood oxygen content is performed while a patient is at rest.
